(19) United States
(12) Patent Application Publication
(10) Pub. No.: US 2008/0153096 A1
(43) Pub. Date: Jun. 26, 2008

(54) CARTRIDGE FOR CONDUCTING DIAGNOSTIC ASSAYS

(75) Inventors: Thomas R. Witty, Tucson, AZ (US); Mark E. Wobken, Orange, CA (US)

Correspondence Address: SHEPPARD, MULLIN, RICHTER & HAMPTON LLP, 2225 EAST BAYSHORE ROAD, SUITE 205, PALO ALTO, CA, 94303, US

(73) Assignee: Vectrant Technologies Inc., Irvine, CA (US)

(21) Appl. No.: 11/933,900

(22) Filed: Nov. 1, 2007

Related U.S. Application Data
(60) Provisional application No. 60/856,534, filed on Nov. 2, 2006.

(51) Int. Cl. C12Q 1/68; C12M 1/00; C12M 1/34; C12M 1/12; C12M 1/40; C12M 1/02; C12M 1/38; G01N 33/53

(52) U.S. Cl. 435/6; 435/287.1; 435/288.7; 435/287.2; 435/287.7; 435/287.3; 435/286.1; 435/7.1; 435/7.9

(57) ABSTRACT

Provided is a cartridge for use in conducting diagnostic assays. The cartridge is configured to maintain sample fluids in a sealed manner and may be used to conduct one or more assays from a single patient sample within a single cartridge. Assays that may be conducted with the cartridge of the present invention include immunoassays and molecular assays. Methods of use for the cartridge and a system for using of the cartridge in combination with a module are also provided.

### CROSS-REFERENCE TO RELATED APPLICATIONS

This application claims the benefit under 35 U.S.C. § 119(e) to U.S. Provisional Patent Application No. 60/856,534 filed on Nov. 2, 2006, which is incorporated herein by reference in its entirety.

### FIELD OF THE INVENTION

The present invention relates generally to devices for use in diagnostic assays, and more particularly to a cartridge used for carrying out at least two types of assays on a single patient sample.

### BACKGROUND OF THE INVENTION

The ability to quantitatively measure a wide variety of physiologically active compounds is important as an adjunct to diagnosis and therapy. The medical industry has become increasingly dependent on the ability to carry out diagnostic assays that measure various physiological parameters in a biological sample, such as for example, antigen levels, antibody levels, protein and/or peptide levels, viral load, bacterial infection, genomic sequences, and the presence of legal and/or illegal active agents in an individual's system. Examples of biological samples that are required in order to run the diagnostic assays include without limitation, blood, blood plasma, epidermal cells, mucosal cells, urine, and saliva.

Diagnostic assays of biological samples have traditionally been performed in sophisticated laboratories that have a substantial investment in equipment and human resources. Recently, there has been an increasing focus on being able to carry out diagnostic assays in smaller and/or less sophisticated laboratories that do not require the degree of investment required of the larger laboratories.

In order for smaller and/or less sophisticated laboratories to perform the diagnostic assays required by the medical industry, there is a need in the art for cost effective equipment that may be used to carry out more than one diagnostic assay on a single biological sample, such as for example, an immunoassay and a molecular assay.

### SUMMARY OF THE INVENTION

The present invention overcomes this need in the art by providing a cartridge that may be used to conduct two separate diagnostic assays on a single biological sample.

In one aspect of the invention, there is provided a cartridge configured to perform at least two assays comprising: an input port for receiving a fluid sample; at least one immunoassay fluid passageway for conducting an immunoassay; and at least one molecular assay fluid passageway for conducting a molecular assay, wherein the cartridge is placed into a module with detection means that record results of the immunoassay and the molecular assay.

In another aspect of the invention, there is provided a method of performing an immunoassay and a molecular assay on a single sample comprising the steps of: obtaining a sample from a patient; and transferring the sample to a cartridge comprising at least one immunoassay fluid passageway for conducting the immunoassay and at least one molecular assay fluid passageway for conducting the molecular assay, wherein the cartridge is placed into a module with detection means that record results of the immunoassay and the molecular assay.

In a further aspect of the invention, there is provided, a system for conducting at least two assays on a single patient sample, comprising: a cartridge comprising an input port for receiving a sample, at least two fluid passageways for performing the at least two assays, and at least one pressure port for regulating the movement of fluid through the fluid passageways; and a module for housing the cartridge comprising detection means for recording results of the at least two assays.

In one embodiment of the invention, the detection means are selected from the group consisting of fluorescence, absorbance, luminescence, electrochemical changes, and magnet pull.

In another embodiment of the invention, the at least two assays are independently selected from the group consisting of immunoassays, molecular assays, electrolyte assays, coagulation assays, routine chemistry assays, and hematology assays.

In a further embodiment of the invention, the at least two fluid passageways are be independently in communication with at least two waste chambers.

In yet another embodiment of the invention, the cartridge comprises at least two chambers for storing reagents and/or buffers required to carry out the at least two assays.

In still a further embodiment of the invention, the module is equipped with at least one actuator for facilitating movement of the sample and reagents through at least one of the at least two fluid passageways.

In another embodiment of the invention, the at least two chambers are subject to independent temperature control via an energy source, wherein energy source is a heating source selected from the group consisting of a heater, a heat pump, a light energy source, and microwaves or a cooling source selected from the group consisting of recirculating coolers, thermoelectric coolers, liquid-to-liquid cooling systems, and ambient cooling systems.

In a further embodiment of the invention, the sample is blood taken from a patient by either venipuncture or traditionally using a needle and primary collection tube. Where the sample is taken via venipuncture, the venipuncture tube is placed directly into the input port of the cartridge, wherein the input port is in communication with the at least one immunoassay fluid passageway and the at least one molecular assay fluid passageway. Where the sample is taken from the patient using a primary collection tube, the blood is taken from the primary collection tube and inserted into an input port of the cartridge using a pipette.

In yet another embodiment of the invention, where the patient sample is blood, the cartridge further comprises a filter to separate plasma from other blood products, wherein the at least two assays may be performed on the plasma.

In still a further embodiment of the invention, the at least one immunoassay fluid passageway comprises a conjugate, which may selected from the group consisting of labeled antibodies, labeled nucleic acids, and labeled particle, the latter of which may be selected from the group consisting of magnetic particles, paramagnetic particles, fluorescent particles, resins, enzymes, and fluorophores.

In another embodiment of the invention, the conjugate lines the interior wall of the at least one immunoassay fluid passageway.

In a further embodiment of the invention, the at least one immunoassay fluid passageway comprises an absorbent pad comprising capillary pumps.

In yet another embodiment of the invention, the at least one immunoassay fluid passageway comprises a capture zone comprised of a permeable material, which may be selected from the group consisting of nitrocellulose, cellulose acetate, hydroxypropylcellulose, phenolic resins, and textured polypropylenes.

In still a further embodiment of the invention, the at least one molecular assay fluid passageway is in communication with at least two chambers, one of which may be a chamber for storing reagents and/or buffers required to carry out the molecular assay and another of which may be a purification chamber where the sample is mixed with the reagents and/or buffers.

In another embodiment of the invention, the cartridge further comprises one or more valves for facilitating communication between the at least one molecular assay fluid passageway and the at least two chambers. The valves may be selected from the group consisting of rotary valves, one-way valves, and two-way valves.

In a further embodiment of the invention, the module is equipped with at least one actuator for facilitating movement of the sample through the molecular assay fluid passageways.

In yet another embodiment of the invention, the at least one actuator is be mixing actuator that facilitates mixing of the sample with reagents required to carry out the molecular assay, wherein the mixing actuator may be selected from a piezo-electric device and an ultrasound device.

In still a further embodiment of the invention, the module further comprises a magnetic actuator for controlling magnetic particles used for purifying nucleic acids for the molecular assay.

In another embodiment of the invention, the cartridge further comprising at least two independently controllable temperature chambers, wherein the molecular assay is a thermal cycling amplification assay, and further wherein, the at least two temperature chambers are coupled to an energy source that generates heat.

In a further embodiment of the invention, the cartridge further comprises at least one pressure port in communication with the immunoassay fluid passageways and the molecular assay fluid passageways, wherein the pressure port may be coupled to a pressure source selected from pneumatic pressure and hydroelectric pressure.

In yet another embodiment of the invention, the cartridge further comprises a waste chamber in communication with the immunoassay fluid passageway and a waste chamber in communication with the molecular fluid passageway.

Other aspects and embodiments of the invention will become apparent to those of skill in the art through a reading of the following description that follows including the appended claims and through practice of the invention.

### BRIEF DESCRIPTION OF THE DRAWINGS

FIG. 1 is a schematic diagram of the top of a cartridge of the present invention.

FIG. 2 is a schematic diagram of the base of a cartridge of the present invention

FIG. 3 is a schematic diagram of the cartridge of the present invention in communication with two actuators of the module that houses the cartridge.

### DETAILED DESCRIPTION OF THE INVENTION

Set forth below is a description of what are currently believed to be the preferred embodiments and best examples of the claimed invention. Any alternates or modifications in structure, function, or purpose are intended to be covered by the claims of this application.

The present invention will be described with reference to the figures. It is to be understood that the figures are meant only to assist in the description of the invention and are not intended to limit the scope of the invention. As used in this specification and the appended claims, the singular forms “a,” “an,” and “the” include plural referents unless the context clearly dictates otherwise.

As used herein, the term “conjugate” is meant to refer to any material that may bind with a sample. Examples of conjugates that may be used with the present invention include without limitation, antigen-antibody conjugate pairs, complementary nucleic acid base pairs, and labeled particles (e.g., magnetic particles, paramagnetic particles, fluorescent particles, resins, enzymes, and fluorophores). To facilitate detection of the conjugates, it is preferred that conjugates used with the present invention are labeled with an appropriate label, such as a fluorescent dye or fluorophore.

The term “primary collection tube” is meant to refer to a traditional tube used for obtaining blood from a patient.

The term “venipuncture tube” is meant to refer to a vacuum tube used for obtaining blood from a patient.

The cartridge of the present invention is intended to be a disposable cartridge that keeps the sample fluid fully sealed within the cartridge at all times during the running of the assays. Upon completion of the assays, the cartridge is intended to be disposed of in an appropriate biohazard disposal waste container. At no time during the running of the assays with the cartridge of the present invention is the operator exposed to the sample or to the reagents used to run the sample.

The cartridge of the present invention is designed to be adapted to perform any two types of assays, which may be independently selected from immunoassays, molecular assays, electrolyte assays, coagulation assays, routine chemistry assays, and hematology assays.

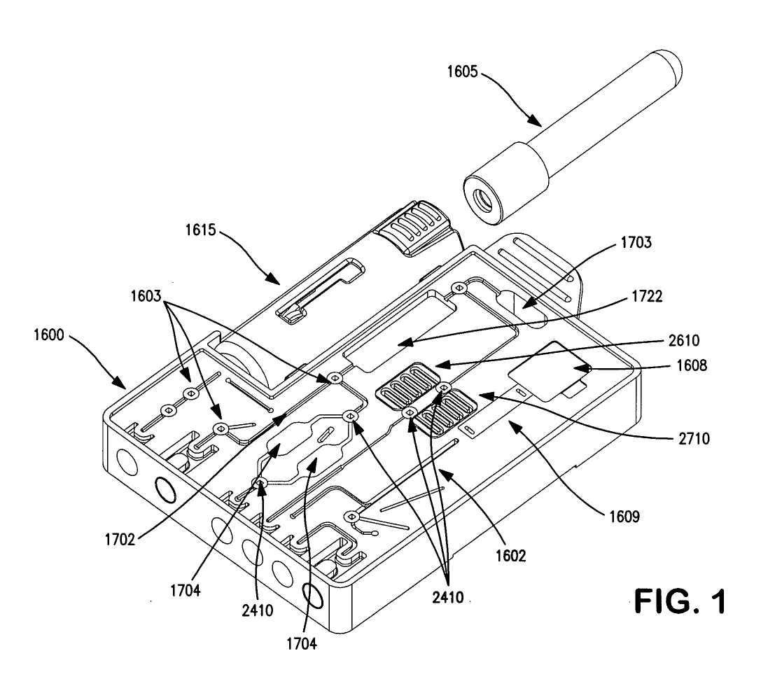

FIGS. 1-3 show a cartridge 1600 that is configured to perform an immunoassay and a molecular assay. It is to be understood that the cartridge shown in FIGS. 1-3 is merely illustrative of an exemplary use of the cartridge of the present invention and is not meant to be limiting with respect to the types of assays that the cartridge of the present invention may perform.

With reference to FIG. 1, in one embodiment of the invention, the cartridge 1600 includes an input port 1615 having an internal cavity that is sized to removably receive a sample tube 1605, such as for example a VACUTAINER® sample tube (Becton Dickinson and Co., Franklin Lakes, N.J., USA). It is to be understood that the cartridge of the present invention is not limited to use with a sample tube, but rather, may be configured to accept sample directly from a primary collection tube using a device, such as for example, a pipette.

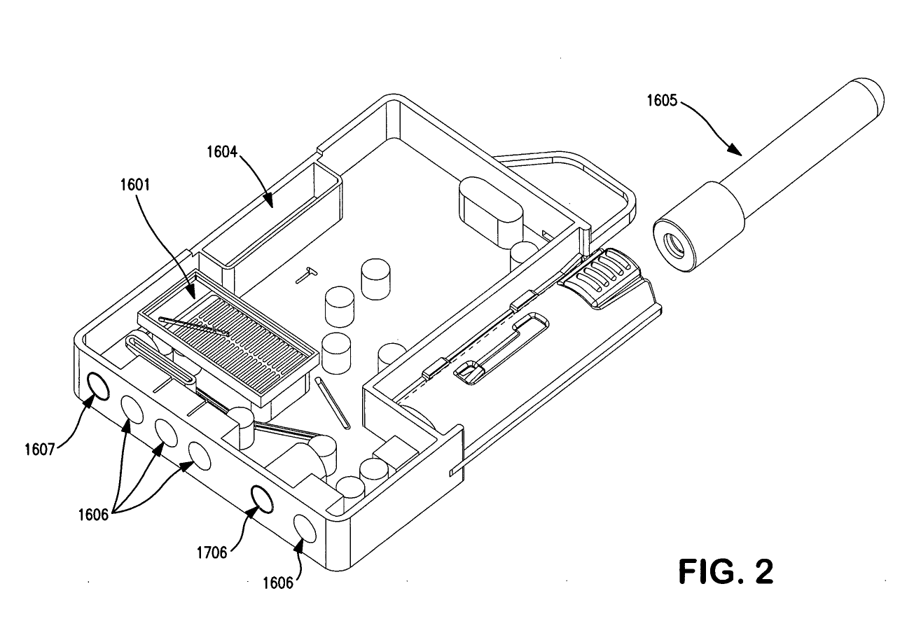

With reference to FIGS. 1 and 2, sample is pushed from the sample container 1605 in the input port 1615, into fluid passageways 1602, 1702 contained within the cartridge by way of pressurization via one or more pressure ports 1606. The fluid passageways of the cartridge facilitate the performance of the at least two assays. In this respect, some fluid passageways are configured to perform one of the at least two assays while other fluid passageways are configured to perform another of the at least two assays. For example, with reference to FIG. 1, upon entry of the fluid into the cartridge 1600 (which will be described in more detail below), the cartridge splits the sample and sends a portion of the sample to fluid passageways 1602 for immunoassay analysis and another portion of the sample to fluid passageways 1702 for molecular analysis.

As indicated above, sample is introduced into the cartridge of the present invention by way of pressure, which is applied to one or more of the pressure ports. Where the cartridge of the present invention is equipped with a single pressure port, a pressure source (not shown) will be attached to the single pressure port. Where the cartridge of the present invention is equipped with multiple pressure ports (as is shown in FIG. 2), either a single pressure source (not shown) or multiple pressure sources may be attached to the pressure ports. Where a single pressure source is used, the source should be capable of applying pressure independently to each pressure port.

Pressure sources that may be used with the cartridge of the present invention include without limitation, pneumatic pressure or hydroelectric pressure.

With reference to FIGS. 1 and 2, where the cartridge is equipped with multiple pressure ports, one or more of the pressure ports may serve to regulate the flow of sample from the input dock 1615 into and through the fluid passageways 1602, 1702 of the cartridge with the remaining pressure ports serving to regulate the flow of buffers or other reagents from buffer chambers 1607, 1706 into and through the fluid passageways 1602, 1702 of the cartridge. FIG. 2 shows a cartridge with four pressure ports. In the embodiment shown in FIG. 2, the pressure port 1606 closest to the input port 1615 may serve to regulate the flow of sample fluid from the input port 1615 into the fluid passageways 1602, 1702 and the remaining three pressure ports 1606 may serve to regulate the flow of buffers housed in the immunoassay buffer chamber 1607 and the molecular assay buffer chamber 1706.

Passageways of the cartridge that may be used to carry out immunoassays include, without limitation: filtration passageways (e.g., using commercial filtration media, such as, polycarbonate membranes and/or glass fibers); lateral flow passageways (e.g., for pregnancy tests); serology binding assay passageways (e.g., for antibody binding assays); non-serology binding assay passageways (e.g., for protein and/or peptide binding assays); incubation passageways (e.g., for use in testing for microbes); and electrochemical passageways (e.g., U.S. Pat. No. 6,551,495 to Porter and Bradley).

Passageways of the cartridge that may be used to carry out molecular assays include, without limitation: amplification passageways to carry out assays such as for example, polymerase chain reaction (PCR) (with DNA as the starting material), reverse transcriptase PCR (RT-PCR) (with RNA as the starting material), nucleic acid sequence-based amplification (NASBA), and strand displacement amplification (SDA); isothermal amplification passageways to carry out assays such as for example, helicase dependent amplification (Biohelix, Beverly, Mass.); signal amplification passageways to carry out assays such as for example, bDNA signal amplification (Bayer Healthcare LLC, Berkeley, Calif.)); RNA expression profiling passageways (e.g., to identify RNA transcripts); and DNA genotyping passageways (e.g., to identify single nucleotide polymorphisms (SNPs)).

Other fluid passageways that may be used with the cartridge of the present invention will be appreciated by those of skill in the art.

Commonly owned U.S. Patent Publication No. 2006/016558 A1 to Witty and Castanon, which is incorporated herein by reference in its entirety, is directed to a cartridge that performs a single assay and describes fluid passageways that may be adapted into a cartridge of the present invention in order to perform immunoassays such as those set forth above.

The cartridge of the present invention 1600 will be configured to include as many chambers as are needed to run the at least two assay. For example, a cartridge of the present invention will typically include one or more of the following: waste chambers, buffer chambers, reagent chambers, mixing and/or purification chambers, and temperature chambers. Exemplary chambers that may be used with the cartridge of the present invention will be described with reference to the figures. The chambers as described are meant only to illustrate possible chambers that may be used with the cartridge of the present invention and are not meant to be limiting to the use or placement of the chambers.

With reference to FIG. 2, waste chamber 1604, which is shown on the base of the cartridge, communicates with the immunoassay fluid passageway 1602 for receiving waste material, such as for example, cellular material from the immunoassay. With reference to FIG. 1, waste chamber 1703 communicates with molecular assay passageway 1703 for receiving waste material, such as for example, used wash buffer and lysed sample from the molecular assay.

With reference again to FIG. 2, buffer chamber 1607 houses buffers for the immunoassay while buffer chamber 1706 houses buffers for the molecular assay. As described previously, the pressure source will serve to push the buffers from the buffer chambers 1607, 1706 into their appropriate fluid passageways 1602, 1702; thus, buffers from buffer chamber 1607 will be pushed into fluid passageway 1602 while buffers from buffer chamber 1706 will be pushed into fluid passageway 1702. In one embodiment of the invention, the buffers in 1607 and 1706 are wash buffers, which are pushed by the pressure source, into the fluid channels 1602, 1702 immediately behind the sample and in another embodiment, the wash buffer does not immediately follow the sample; rather, there is a gap, such as for example, an air gap, between the sample and the wash buffer.

With reference to FIG. 1, purification chamber 1722 is where the sample is mixed with a binding material, such as an antibody, nucleic acid, resin, or paramagnetic particle; reagent chamber 1704 houses reagents, such as lysis buffer and amplification reagents that are used for the molecular assay (described in further detail below); and temperature chambers 2610 and 2710 are used for thermal cycling amplification reactions, such as for example polymerase chain reaction (PCR) assays. Depending on the type of thermal cycling reaction that is to be run with the cartridge of the present invention, the cartridge may be configured with two temperature chambers (as is shown in FIG. 1) or with three temperature chambers (as may be required to run three temperature PCR assays; see Examples 1, 2, and 3).

It is to be understood that the cartridge of the present invention may include any additional chambers that are needed to carry out specific assays. Similarly, to run a particular assay, a cartridge may not require all of the chambers that are described above. For example, for isothermal amplification reactions, it will be appreciated that the cartridge may require fewer temperature chambers.

Each of the individual chambers of the cartridge of the present invention may be configured to be subject to individual temperature controls. As will be appreciated by those of skill in the art, to run thermal cycling reactions, each of the temperature chambers will require individual temperature controls to keep the temperature of the chambers constant; however, it should be appreciated that chamber temperature control need not be limited to the temperature chambers. For example, there may be some assays where it may be desirable to keep the temperature of a buffer chamber or a reagent chamber at a certain temperature. The temperature of a chamber may be regulated by using any appropriate energy source. For example, chambers may be heated by coupling the chambers to a heater, a heat pump, a light energy source, or microwaves. Chambers may be cooled by coupling the chambers to recirculating coolers, thermoelectric coolers, liquid-to-liquid cooling systems, and ambient cooling systems. Other means for heating or cooling the chambers will be appreciated by those of skill in the art.

As shown in FIG. 1, the cartridge 1600 further includes one or more valves 1603 and 2410 for isolating or trapping fluid within the fluid passageways 1602, 1702 and/or the chambers 1704, 1722. Examples of valves that may be used with the cartridge include any suitable valve, such as for example, rotary valves, one-way valves, or two-way valves,

The cartridge 1600 of the present invention is intended to be placed into a module equipped with detection means (not shown) that will process the assays upon coupling with the cartridge 1600. Detection means that may be found within the module may include without limitation means for detecting any of the following: fluorescence, absorbance, luminescence, electrochemical changes, and magnetic pull. In one example, the module may be equipped with a photo-optic array, which may be used to detect optical changes in the sample as it runs through the fluid passageways 1602, 1702.

The cartridge of the present invention is designed to facilitate the configuration of fluid passageways that will maximize the efficiency and accuracy of the assays.

For example, in one embodiment of the invention, which is depicted schematically in FIG. 1 and which may be useful for running immunoassays, fluid passageways 1602 may equipped with an absorbent pad 1608 that facilitates the lateral flow of the sample through fluid passageways 1602. The absorbent pad 1608 may include one or more capillary structures that act as capillary pumps to facilitate efficient and smooth flow of the sample through fluid passageways 1602. Fluid passageways 1602 may be further equipped with a capture zone 1609 for capturing sample and wash buffer. In one embodiment of the invention, the capture zone is comprised of a permeable material, which may be a bibulous or non-bibulous material. Examples of permeable bibulous materials that may be used with the cartridge of the present invention include without limitation nitrocellulose, cellulose acetate, and hydroxypropylcellulose. Examples of permeable non-bibulous materials that may be used with the cartridge of the present invention include without limitation phenolic resins and textured polypropylenes or other engineered plastic. Upon completion of the immunoassay, the detection means of the module will record the results of the immunoassay. For example, in another embodiment of the invention, the fluid passageway is designed with bound fluorescent labeled antibody. The antibodies may be attached directly to the fluid passageways 1602 or they may be bound to a membrane that is attached to the walls of the fluid passageways 1602. As the sample passes through the fluid passageway 1602, antigen in the sample binds with the antibody. Upon completion of the assay, the module will be able to record the fluorescence of the antigen-antibody conjugate pair within the capture zone 1609. As indicated above, it is to be understood that the immunoassay is not limited to antigen-antibody conjugate pairs, but may include any suitable conjugate pair, such as complementary nucleic acid base pairs and labeled particles selected from the group consisting of magnetic particles, paramagnetic particles, fluorescent particles, resins, enzymes, and fluorophores.

In a further embodiment of the invention, the cartridge is equipped with a filter 1601 (FIG. 2), which may be used to prepare the sample for immunoassay and/or molecular analysis. For example, where the sample is blood, filter 1601 may be used to filter the blood so that only the plasma may run through the fluid pathways 1602 and 1702.

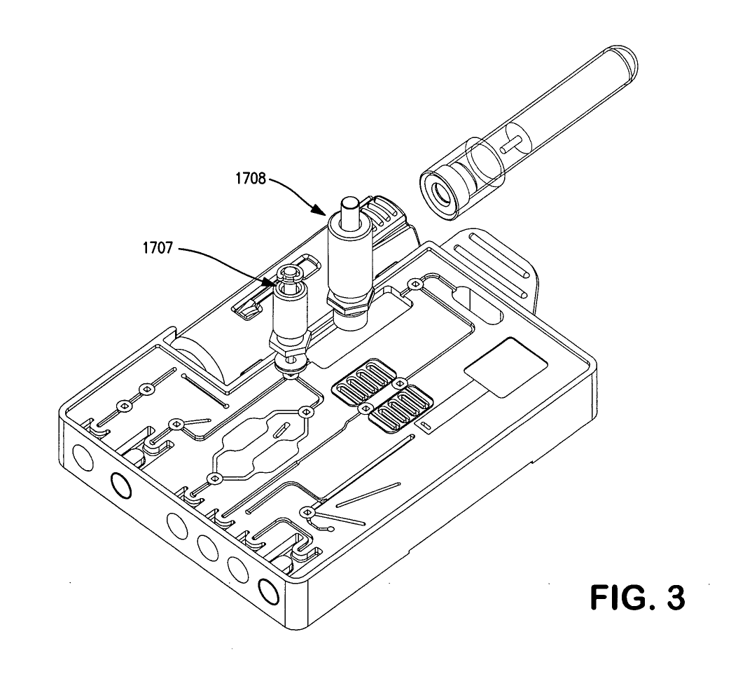

In another embodiment of the invention, which is depicted schematically in FIGS. 1 and 3 and which may by useful for running molecular assays, the module that houses the cartridge 1600 is configured with hardware that facilitates the mixing of sample with various reagents and buffers. For example, the module may include one or more actuators 1707, 1708 that communicate with at least one of the fluid passageways 1702 and/or chambers 1722 of the cartridge 1600. In one embodiment, the actuators are a device, such as a piezo-electric device or an ultrasound device that imparts energy into the fluid passageways 1702 and/or chambers 1722 in order to mix the contents therein. For example, the actuators 1707, 1708 can be used to mix the sample and conjugate in the purification chamber 1722. In order to be able to enter and exit the fluid passageways 1702 and/or chambers 1722, the actuators 1707, 1708 may be equipped with means for moving the actuators, such as for example, a solenoid or a pneumatic actuator.

In another embodiment of the invention, the module includes two actuators, a mixing actuator 1707 and a magnetic actuator 1708, which are in communication with the cartridge of the present invention 1600. It is to be understood that the actuators shown schematically in FIG. 3 are not part of the cartridge, but rather, are part of the module (not shown), which is in communication with the cartridge 1600. The relationship between the mixing 1707 and magnetic actuators 1708 on the module and the fluid passageways 1702 and chambers 1722 of the cartridge 1600 will be described with reference to an embodiment of the invention where the cartridge 1600 is used to conduct a nucleic acid purification assay using paramagnetic particles, which are housed in a purification chamber 1722 of the cartridge 1600.

The use of paramagnetic particles for the purification of nucleic acids, such as DNA and RNA, is known in the art and is described in the following patents: U.S. Pat. No. 6,433,160 to Collis et al; U.S. Pat. No. 5,705,628 to Hawkins; U.S. Pat. No. 5,695,946 and U.S. Pat. No. 5,491,068 both to Benjamin et al.; U.S. Pat. No. 5,536,644 to Ullman et al.; U.S. Pat. No. 5,512,439 to Homes et al.; U.S. Pat. No. 5,395,688 to Wang et al.; U.S. Pat. No. 5,232,782 to Charmot; U.S. Pat. No. 4,774,265 to Ugelstad et al.; U.S. Pat. No. 4,695,393 to Whitehead et al.; U.S. Pat. No. 4,672,040 to Josephson; U.S. Pat. No. 4,230,685 to Senyei et al.; U.S. Pat. No. 4,141,687 to Forrest et al.; U.S. Pat. No. 3,970,518 to Giaever; EP Publication Nos. 0 444 120 B1 and 0 446 260 B1 both to Homes et al.; and PCT Publication No. WO 96/18731 to Deggerdal et al. Commercially available paramagnetic particles that may be used with the present invention may be obtained from Serodyne or Promega Corp., Merck, Bangs. MagneSil® paramagnetic particles and streptavidin MagneSphere® paramagnetic particles both products of Promega Corporation (Madison, Wis., USA). Preferred paramagnetic particles for use with the cartridge of the present invention are pan-specific paramagnetic particles (i.e., paramagnetic particles that are reactive with a nucleic acid from any species and/or disease state).

Within the context of the present invention, the paramagnetic particles will be housed in the purification chamber in any suitable form, such as for example, a liquid state, a solid state, a suspended state, or in a lyophilized cake.

With reference now to the nucleic acid purification assay using paramagnetic particles, the sample flows through the molecular assay fluid passageways 1702 (FIG. 1) where a valve of reagent chamber 1704 is opened to release lysis buffer 1704, which mixes with the sample causing lysis of the sample. The lysed sample then proceeds to the purification chamber 1722 where it mixes with the paramagnetic particles that are housed in the purification chamber 1722. Upon entry of the lysed sample buffer into the purification chamber 1722, the mixing actuator 1707 is moved into a position at or near to the purification chamber 1722 where it is activated to initiate mixing of the contents of the purification chamber 1722 via agitation. The agitation and mixing of the contents of the purification chamber 1722 will cause the paramagnetic particles to bind to nucleic acids in the lysed sample.

Next, a wash cycle is initiated. To begin the wash cycle, the mixing actuator 1707 moves away from the purification chamber 1722 and the magnetic actuator 1708 takes its place where it is activated to hold the paramagnetic particles in place on the top inside wall of the purification chamber 1722. Wash buffer, which is released from buffer chamber 1706 is then forced into the purification chamber 1722 by way of pressurization through pressure port 1606 and rinses the contents of the purification chamber 1722 while the magnet actuator 1708 holds the paramagnetic particles in place. Upon completion of the wash cycle, the wash buffer along with the sample is forced into waste chamber 1703. The wash cycle can be repeated multiple times. As is known to those of skill in the art, thorough washing of a sample can frequently reduce background noise that may interfere with the analytical reading of a sample. The eluate of the final wash will not be forced into the waste chamber 1703 but rather, will be retained in the purification chamber 1722 where it will be used to initiate the amplification portion of the nucleic acid purification assay.

The amplification portion of the nucleic acid purification assay begins with the opening of a valve of reagent chamber 1704 to release amplification reagents from reagent chamber 1704 into the purification chamber 1722, where the amplification reagents mix with the eluate left in the purification chamber 1722. In order for the module to be able to detect the progress of the amplification reaction and the reaction products, the amplification reagents should include detectable label, such as a dye or a fluorophore. The magnetic actuator 1708 then releases the paramagnetic particles and moves away from the purification chamber 1722 and the mixing actuator 1707 takes its place at or above the purification chamber 1722 where it is activated to cause agitation and mixing of the paramagnetic particles and the amplification reagents.

Next, the mixing actuator 1707 moves away from the purification chamber 1722 and the magnetic actuator returns to the purification chamber 1722 where it is activated to hold the paramagnetic particles in place on the top wall of the purification chamber. The eluate is then transferred to a first temperature chamber 2610 that is coupled to an energy source (not shown), which can be used to heat the chamber and incubate the mixture. For thermal cycling reactions, such as PCR, two to three temperature chambers will be required to run the amplification reactions, with each of the temperature chambers subject to independent temperature controls. With reference to FIG. 1, which schematically depicts a cartridge equipped with two temperature chambers 2610 and 2710, for thermal cycling reactions, the eluate will be transferred between temperature chambers 2610 and 2710, where the sample is incubated for an appropriate length of time, until the amplification reaction is complete. It is to be understood that the cartridge of the present invention may be configured to house as many temperature controlled zones as necessary to carry out thermal cycling and/or isothermal reactions. For example, for isothermal reactions, only one temperature chamber may be necessary to run the amplification reaction. For some PCR and RT-PCR amplifications, three or four temperature chambers may be required in order to efficiently run the hot start, denaturation, annealing, and extension reactions required of the amplification reactions (see Examples 1, 2, and 3). For other reactions, it may be necessary to design a cartridge with more than four temperature chambers.

During the course of the amplification reaction, detection means in the module may record the progress of the reaction by measuring a signal from the reaction products. For example, where the amplification reagents include a label, the module will record a signal each time an amplification cycle is complete. In this way, the progress of the reaction can be tracked by measuring the intensity of the signal. The cartridge of the present invention may be designed so that the amplified sample remains sealed in the cartridge and is discarded along with the disposable container or alternatively, the cartridge may be designed so that the amplified sample may be removed for further testing.

It is to be understood, that the nucleic acid purification assays that are conducted with the cartridge of the present invention are not limited to paramagnetic bead purification assays, but rather, the cartridge of the present invention may be adapted to conduct any type of nucleic acid purification assay. For example, the cartridge of the present invention may be used to run purification assays using ion-exchange resins (e.g., Qiagen Anion-Exchange Resin Qiagen, Valencia, Calif.) and/or non-magnetic functionalized particles that are specific to DNA/RNA. Where non-magnetic purification means are used, it is to be understood that a magnetic actuator, such as the one described above, may not be necessary and that the molecular assay may be run with a single mixing actuator, alone or in combination with one or more actuators or other hardware devices that will facilitate the running of the molecular assay.

All patents and publications mentioned herein are incorporated by reference in their entireties.

It is to be understood that while the invention has been described in conjunction with the preferred specific embodiments, the foregoing description as well as the examples that follow are intended to illustrate and not limit the scope of the invention. In this respect, the scope of the appended claims should not be limited to the description of the embodiments described herein. Other aspects, advantages and modifications within the scope of the invention will be apparent to those skilled in the art to which the invention pertains.

### EXPERIMENTAL

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### Example 1

### HPV Status Determinations

Prior to receiving the GARDASIL® HPV vaccine (Merck & Co., Inc., Whitehouse Station, N.J., USA), the HPV status of a woman must be determined in order to ensure that she is not already infected with HPV. The GARDASIL vaccine protects against HPV, Types 6, 11, 16, and 18.

A gynecologist responsible for administering the GARDASIL® vaccine uses a local lab to run her lab tests for her. Patient's that are eligible for the GARDASIL® vaccine are sent to the lab, which has a phlebotomist on staff, for testing.

The phlebotomist takes blood from the patient via venipuncture with a VACUTAINER® needle and tube. The phlebotomist removes the needle from the VACUTAINER® tube and discards it in a biohazard waste disposal box. The phlebotomist then takes the sealed VACUTAINER® tube and hands it to the laboratory worker for HPV antigen analysis.

The laboratory worker receiving the sample immediately selects an HPV antibody testing cartridge, places it in the module, inserts the VACUTAINER® tube into the input port of the cartridge, and begins the assay by initiating pressure to the cartridge, which pulls sample into the fluid passageways of the cartridge. The laboratory worker controls the assays that are run within the cartridge via the module.

The fluid passageways of the HPV testing cartridge have been primed for HPV analysis by lining the immunoassay fluid passageways with antibodies that bind to HPV antigen types 6, 11, 16, and 18. As HPV is a DNA based virus, the molecular fluid passageways and chambers are equipped with reagents and materials to run a PCR. To facilitate the lateral flow of the HPV antibody assay, the immunoassay fluid passageways are further lined with an absorbent pad equipped with capillary pumps. The PCR assay will use temperatures and times sufficient to run denaturation, annealing, and extension.

At the completion of the HPV antibody immunoassay, the module indicates that the patient has been exposed to, but not infected with HPV. Because the patient is a good candidate for HPV, the molecular analysis is not necessary. The clinician discards the cartridge in a biohazard waste disposal container and inserts a new cartridge into the module fore the next patient

The phlebotomist takes blood from the next patient as for the first patient and the laboratory worker places the sample through the new cartridge for HPV testing. The HPV Ab test indicates that the patient is infected with HPV, Type 6. The molecular assay is run in order to determine the extent of viral load in the patient so that the patient can undergo appropriate anti-viral treatment.

### Example 2

### H5N1 Avian Influenza Status Determinations

Six travelers are sent to a hospital under suspicion of exposure to avian influenza. In order to determine if the patients have been exposed to the deadly H5N1 viral strain, a sample of their blood must be tested at the hospital's lab.

A nurse at the hospital takes a sample of each patient's blood via venipuncture using a VACUTAINER® needle and tube, which has been appropriately labeled for each patent. Upon obtaining the blood sample from the patient, the nurse separates the needles from the tubes, discards the needles in the biohazard waste disposal box, and sends the sealed and labeled tubes down to the hospital lab for analysis. The laboratory worker separates six cartridges (one for each patient) designed to test for the H5N1 strain of avian influenza. The laboratory worker labels each cartridge to match the labels on the venipuncture tubes, places the first cartridge in the module, and applies the sample tube to the cartridge.

The fluid passageways of the H5N1 testing cartridge have been primed for H5N1 analysis by lining the immunoassay fluid passageways of the cartridge with antibodies that bind to the H5N1 antigen. As influenza is an RNA based virus, the molecular fluid passageways and chambers are equipped with reagents and materials to run RT-PCR (reverse transcriptase PCR). To facilitate the lateral flow of the H5N1 antibody assay, the immunoassay fluid passageways are further lined with an absorbent pad equipped with capillary pumps. The RT-PCR assay will use temperatures and times sufficient to run denaturation, annealing, and extension.

The results of the antibody test for the first patient is negative and thus, the laboratory worker does not run the molecular test for that patient. The second and third patients are also found to be negative and thus, no molecular test is run on them as well. The fourth patient is found to be positive for the H5N1 antibody and the molecular test is run on the patient sample in order to determine viral load for appropriate anti-viral therapy. The fifth patient is also found to be negative, but the sixth patient tests positive for H5N1 and thus, the molecular test is run for the sixth patient as well. The results of the test are sent back to the hospital and the first, second, third, and fifth patients are released and the fourth and sixth patients are admitted to the hospital for appropriate treatment.

### Example 3

### HIV Status and Viral Load Determinations

An HIV clinical with limited resources runs daily HIV tests for patients wanting to know their HIV status and daily HIV tests to determine viral load of HIV positive and AIDS patients.

A new patient is presented to the clinic. The clinician running the HIV tests inserts an HIV analysis cartridge into the module and then takes a sample of the patient blood via venipuncture using a VACUTAINER® needle and sample tube. After obtaining the sample, the clinician removes the needle from the tube, discards the needle in a biohazard waste disposal box, inserts the tube into the input port of the cartridge, and applies pressure to push the sample into the immunoassay fluid passageways of the cartridge to run the HIV immunoassay.

The fluid passageways of the HIV testing cartridge have been primed for HIV analysis by lining the immunoassay fluid passageways of the cartridge with antibodies that bind to the p24 antigen. HIV is a retrovirus, which is an enveloped virus with an RNA genome, which replicates via a DNA intermediate. In order to run the molecular part of the assay, the RNA genome is amplified via RT-RCR.

To facilitate the lateral flow of the HIV antibody assay, the immunoassay fluid passageways are further lined with an absorbent pad equipped with capillary pumps. The PCR assay will use temperatures and times sufficient to run denaturation, annealing, and extension.

The results of the antibody test for the patient is positive and thus, the clinician runs the molecular test. The results of the test are provided to the patient and the patient's physician so that the patient may start appropriate anti-viral therapy.

We claim:

1. A cartridge configured to perform at least two assays comprising:
an input port for receiving a fluid sample;
at least one immunoassay fluid passageway for conducting an immunoassay; and
at least one molecular assay fluid passageway for conducting a molecular assay,
wherein the cartridge is placed into a module with detection means that record results of the immunoassay and the molecular assay.

2. The cartridge of claim 1, wherein the detection means are selected from the group consisting of fluorescence, absorbance, luminescence, electrochemical changes, and magnet pull.

3. The cartridge of claim 1, wherein the at least one immunoassay fluid passageway comprises a conjugate.

4. The cartridge of claim 3, wherein the conjugate is selected from the group consisting of labeled antibodies, labeled nucleic acids, and labeled particles.

5. The cartridge of claim 4, wherein the labeled particles are selected from the group consisting of magnetic particles, paramagnetic particles, fluorescent particles, resins, enzymes, and fluorophores.

6. The cartridge of claim 3, wherein the conjugate lines an interior wall of the at least one immunoassay fluid passageway.

7. The cartridge of claim 1, wherein the at least one immunoassay fluid passageway comprises an absorbent pad comprising capillary pumps.

8. The cartridge of claim 1, wherein the at least one immunoassay fluid passageway comprises a capture zone comprised of a permeable material.

9. The cartridge of claim 8, wherein the permeable material is selected from the group consisting of nitrocellulose, cellulose acetate, hydroxypropylcellulose, phenolic resins, and textured polypropylenes.

10. The cartridge of claim 1, wherein the at least one molecular assay fluid passageway is in communication with at least two chambers.

11. The cartridge of claim 10, wherein at least one of the at least two chambers is a chamber for storing reagents and/or buffers required to carry out the molecular assay and another of the at least two chambers is a purification chamber where the sample is mixed with the reagents and/or buffers.

12. The cartridge of claim 11, further comprising one or more valves for facilitating communication between the at least one molecular assay fluid passageway and the at least two chambers.

13. The cartridge of claim 12, wherein one or more valves are selected from the group consisting of rotary valves, one-way valves, and two-way valves.

14. The cartridge of claim 1, wherein the module is equipped with at least one actuator for facilitating movement of the sample through the molecular assay fluid passageways.

15. The cartridge of claim 14, wherein the at least one actuator is a mixing actuator that facilitates mixing of the sample with reagents required to carry out the molecular assay.

16. The cartridge of claim 15, wherein the mixing actuator is selected from a piezo-electric device and an ultrasound device.

17. The cartridge of claim 15, wherein the module further comprises a magnetic actuator for controlling magnetic particles used for purifying nucleic acids for the molecular assay.

18. The cartridge of claim 1, further comprising at least two independently controllable temperature chambers, wherein the molecular assay is a thermal cycling amplification assay.

19. The cartridge of claim 18, wherein the at least two temperature chambers are coupled to an energy source that generates heat.

20. The cartridge of claim 1, further comprising at least one pressure port in communication with the immunoassay fluid passageways and the molecular assay fluid passageways.

21. The cartridge of claim 20, wherein the pressure port is coupled to a pressure source selected from pneumatic pressure and hydroelectric pressure.

22. The cartridge of claim 1, further comprising a waste chamber in communication with the immunoassay fluid passageway.

23. The cartridge of claim 1, further comprising a waste chamber in communication with the molecular fluid passageway.

24. The cartridge of claim 1, wherein the sample is blood and the cartridge further comprises a filter to separate plasma from other blood products.

25. The cartridge of claim 24, wherein the immunoassay and the molecular assay are carried out on the plasma.

26. A method of performing an immunoassay and a molecular assay on a single sample comprising the steps of:
obtaining a sample from a patient; and
transferring the sample to a cartridge comprising at least one immunoassay fluid passageway for conducting the immunoassay and at least one molecular assay fluid passageway for conducting the molecular assay,
wherein the cartridge is placed into a module with detection means that record results of the immunoassay and the molecular assay.

27. The method of claim 1, wherein the detection means are selected from the group consisting of fluorescence, absorbance, luminescence, electrochemical changes, and magnet pull.

28. The method of claim 26, wherein the sample is blood.

29. The method of claim 28, wherein the blood is taken from the patient using a venipuncture tube.

30. The method of claim 29, wherein the venipuncture tube is inserted directly into an input port of the cartridge, wherein the input port is in communication with the at least one immunoassay fluid passageway and the at least one molecular assay fluid passageway.

31. The method of claim 28, wherein the blood is taken from the patient using a primary collection tube.

32. The method of claim 31, wherein the blood is taken from the primary collection tube and inserted into an input port of the cartridge using a pipette, wherein the input port of the cartridge is in communication with the at least one immunoassay fluid passageway and the at least one molecular assay fluid passageway.

33. The method of claim 28, wherein the cartridge further comprises a filter that filters plasma from other blood products, wherein the immunoassay and the molecular assay are carried out on the plasma.

34. The method of claim 26, wherein the at least one immunoassay fluid passageway comprises a conjugate.

35. The method of claim 33, wherein the conjugate is selected from the group consisting of labeled antibodies, labeled nucleic acids, and labeled particles.

36. The method of claim 34, wherein the labeled particles are selected from the group consisting of magnetic particles, paramagnetic particles, fluorescent particles, resins, enzymes, and fluorophores.

37. The method of claim 26, wherein the conjugate lines an interior wall of the at least one immunoassay fluid passageway.

38. The method of claim 26, wherein the at least one molecular assay fluid passageway is in communication with at least two chambers.

39. The method of claim 37, wherein at least one of the at least two chambers is a chamber for storing reagents and/or buffers required to carry out the molecular assay and another of the at least two chambers is a purification chamber where the sample is mixed with the reagents and/or buffers.

40. The method of claim 37, further comprising one or more valves for facilitating communication between the at least one molecular assay fluid passageway and the at least two chambers.

41. The method of claim 39, wherein one or more valves are selected from the group consisting of rotary valves, one-way valves, and two-way valves.

42. The method of claim 26, wherein the module is equipped with at least one actuator for facilitating movement of the sample through the molecular assay fluid passageways.

43. The method of claim 41, wherein the at least one actuator is a mixing actuator that facilitates mixing of the sample with reagents required to carry out the molecular assay.

44. The method of claim 42, wherein the mixing actuator is selected from a piezo-electric device and an ultrasound device.

45. The method of claim 42, wherein the module further comprises a magnetic actuator for controlling magnetic particles used for purifying nucleic acids for the molecular assay.

46. The method of claim 26, further comprising at least two independently controllable temperature chambers, wherein the molecular assay is a thermal cycling amplification assay.

47. The method of claim 45, wherein the at least two temperature chambers are coupled to an energy source that generates heat.

48. The method of claim 26, further comprising at least one pressure port in communication with the immunoassay fluid passageways and the molecular assay fluid passageways.

49. The method of claim 47, wherein the pressure port is coupled to a pressure source selected from pneumatic pressure and hydroelectric pressure.

50. The method of claim 1, further comprising a waste chamber in communication with the immunoassay fluid passageway and a waste chamber in communication with the molecular fluid passageway.

51. A system for conducting at least two assays on a single patient sample, comprising:
a cartridge comprising an input port for receiving a sample, at least two fluid passageways for performing the at least two assays, and at least one pressure port for regulating the movement of fluid through the fluid passageways; and
a module for housing the cartridge comprising detection means for recording results of the at least two assays.

52. The system of claim 51, wherein the detection means are selected from the group consisting of fluorescence, absorbance, luminescence, electrochemical changes, and magnet pull.

53. The system of claim 51, wherein the at least two assays are independently selected from the group consisting of immunoassays, molecular assays, electrolyte assays, coagulation assays, routine chemistry assays, and hematology assays.

54. The system of claim 51, wherein the at least two fluid passageways are independently in communication with at least two waste chambers.

55. The system of claim 51, wherein the cartridge comprises at least two chambers for storing reagents and/or buffers required to carry out the at least two assays.

56. The system of claim 55, further comprising one or more valves for facilitating communication between the at least two fluid passageway and the at least two chambers.

57. The system of claim 56, wherein one or more valves are selected from the group consisting of rotary valves, one-way valves, and two-way valves.

58. The system of claim 1, wherein the module is equipped with at least one actuator for facilitating movement of the sample and reagents through at least one of the at least two fluid passageways.

59. The system of claim 58, wherein the at least one actuator is a mixing actuator that facilitates mixing of the sample with reagents required to carry out at least one of the at least two assays.

60. The system of claim 59, wherein the mixing actuator is selected from a piezo-electric device and an ultrasound device.

61. The system of claim 55, wherein the at least two chambers are subject to independent temperature control via an energy source.

62. The system of claim 61, wherein the energy source is a heating source selected from the group consisting of a heater, a heat pump, a light energy source, and microwaves.

63. The system of claim 61, wherein the energy source is a cooling source selected from the group consisting of recirculating coolers, thermoelectric coolers, liquid-to-liquid cooling systems, and ambient cooling systems.

64. The system of claim 51, wherein the cartridge further comprises at least one pressure port in communication with the at least two fluid passageways.

65. The system of claim 64, wherein the at least one pressure port is coupled to a pressure source selected from pneumatic pressure and hydroelectric pressure.

66. The system of claim 51, wherein the sample is obtained from the patient using a venipuncture tube, wherein the venipuncture tube is inserted directly into the input port.

67. The system of claim 51, wherein the sample is obtained from the patient using a primary collection tube, wherein the sample taken from the primary collection tube and inserted into the input port of the cartridge using a pipette.

68. The system of claim 51, wherein the patient sample is blood and the cartridge further comprises a filter to separate plasma from other blood products

69. The system of claim 68, wherein the at least two assays are performed on the plasma.
